(19) United States
(12) Patent Application Publication
(10) Pub. No.: US 2024/0198035 A1
(43) Pub. Date: Jun. 20, 2024

(54) MENTAL AROUSAL LEVEL REGULATION SYSTEM AND METHOD

(71) Applicants: NANYANG TECHNOLOGICAL UNIVERSITY, Singapore (SG); NATIONAL UNIVERSITY OF SINGAPORE, Singapore (SG)

(72) Inventors: Cuntai GUAN, Singapore (SG); Tih Shih LEE, Singapore (SG); Yi DING, Singapore (SG); Neethu ROBINSON, Singapore (SG); Shi Hui POON, Singapore (SG); Xiangting Bernice LIN, Singapore (SG); Pinar BILGIN, Singapore (SG); Qiuhao ZENG, Singapore (SG); Aung Aung PHYO WAI, Singapore (SG); Jonathan Zhi Han TAN, Singapore (SG); Natalie Li Wun CHIN, Singapore (SG)

(21) Appl. No.: 18/557,090

(22) PCT Filed: Apr. 22, 2022
(86) PCT No.: PCT/SG2022/050243

§ 371 Date: Oct. 25, 2023

(30) Foreign Application Priority Data
May 12, 2021 (SG) 10202104950T

(51) Int. Cl. A61M 21/02; A61M 21/00; G06F 3/01

(52) U.S. Cl. CPC A61M 21/02; G06F 3/015; A61M 2021/0027; A61M 2021/0083; A61M 2230/10

(57) ABSTRACT

A system and method for mental arousal level regulation of a subject. The system includes at least one sensor and an interface configured to provide a virtual object in a virtual reality environment. A controller is configured to perform a method of mental arousal level regulation including: providing at least one stimulus in the virtual reality environment for a time duration; and acquiring bioelectric signals from the subject via the at least one sensor concurrently with the providing of the at least one stimulus. Based on the bioelectric signals, a mental arousal score for the subject for the time duration is determined. The controller is configured to controllably vary at least one of the virtual object and the at least one stimulus in response to the mental arousal score.

The present application claims priority to the Singapore patent application no. 10202104950T, the entire disclosure of which is incorporated herein by reference.

### TECHNICAL FIELD

The present disclosure relates to the field of bioelectric signal-based therapeutics, and more particularly to systems and methods for regulating a mental state of arousal.

### BACKGROUND

Patients with generalized mental arousal disorders may exhibit physical mental arousal symptoms (such as tachycardia and tremor) and other psychological symptoms, including restlessness, fatigue, difficulty in concentrating, irritability, and disturbed sleep. In traditional therapy, patients generally rely on therapists to help them find ways to deal with stressful situations, for example, by talking over and analyzing past stressful events, avoiding stress-inducing situations, engaging in more physical exercise, etc.

### SUMMARY

In one aspect, the present disclosure provides a system for mental arousal level regulation of a subject, the system comprising: at least one sensor; an interface configured to provide a virtual object in a virtual reality environment, the virtual object and the virtual reality environment being perceptible by the subject; a controller in operable signal communication with the at least one sensor and the interface, the controller being configured to perform a method of mental arousal level regulation including: providing at least one stimulus in the virtual reality environment for a time duration; acquiring bioelectric signals from the subject via the at least one sensor concurrently with the providing of the at least one stimulus; based on the bioelectric signals, determining a mental arousal score for the subject for the time duration; and responsive to the mental arousal score, controllably varying at least one of the virtual object and the at least one stimulus.

The system as described above, in which the controller comprises a machine learning model configured to predictively determine the mental arousal score, and wherein the machine learning model is personalized according to bioelectric signals acquired exclusively from the subject. The machine learning model may comprise a classification model trained using feature segments originating from the bioelectric signals acquired exclusively from the subject. The controller may be configured to controllably vary a property of the virtual object such that the property tracks the mental arousal score. At least a part of the virtual object may be configured to be in motion, the motion being characterized by a quantitative property perceptible by the subject, such that the quantitative property changes correspondingly with the mental arousal score of the subject.

The controller may be configured to controllably vary the at least one stimulus provided in the virtual reality environment by switching between providing a first stimulus and providing a second stimulus. The controller may be configured to determine respective mental arousal scores for the first stimulus and the second stimulus based on the bioelectric signals from the subject, and wherein the respective mental arousal scores are indicative of different mental arousal levels with respect to the subject. In one embodiment, with respect to the subject, the first stimulus is an arousal-inducing stimulus and wherein the second stimulus is a relaxation-inducing stimulus. In another embodiment, with respect to the subject, the first stimulus is a relaxation-inducing stimulus and wherein the second stimulus is an arousal-inducing stimulus. The controller may be configured to switch from providing the first stimulus to providing the second stimulus after providing the first stimulus for a first duration. The first duration may be at least a predetermined duration of time.

The controller may be configured to determine the mental arousal score based on the bioelectric signals acquired exclusively from the subject. The controller may be configured to determine an arousal regulation index based on the bioelectric signals, the arousal regulation index being indicative of a level of mental arousal induced in the subject. The arousal regulation index may be configured to be a quantitative measure of the subject's ability to regulate the subject's own level of mental arousal. In response to the controller finding the arousal regulation index of the subject to be below a threshold value, the controller may be configured to increase the first duration by an additional duration. The controller may be configured to determine a mental arousal regulation index based on a number of instances when the mental arousal score is above an upper threshold or below a lower threshold.

The system according to any described above, in which the controller is further configured to provide an audio feedback responsive to the mental arousal score, and wherein the audio feedback is configured to direct the subject towards changing the mental arousal score towards a preferred range of values.

According to another aspect, the present disclosure provides a method of regulating a mental arousal level of a subject. The method comprises: providing at least one stimulus in a virtual reality environment for a time duration; acquiring bioelectric signals from the subject via at least one sensor concurrently with the providing of the at least one stimulus; based on the bioelectric signals, determining a mental arousal score for the subject for the time duration; and controllably varying at least one of a virtual object and the at least one stimulus, the controllably varying being responsive to the mental arousal score, at least one sensor, wherein the virtual object is provided in the virtual reality environment, the virtual object and the virtual reality environment being configured to be perceptible by the subject according to any of the system described above.

According to yet another aspect, the present disclosure provides a computing device comprising: a controller; a speaker coupled to the controller; an interface coupled to the controller; and a non-transitory computer readable medium having stored thereon computer implementable instructions executable by the controller, the controller being configured to perform a method of mental arousal level regulation including: providing at least one stimulus as a virtual reality environment via the interface for a time duration, the virtual reality environment being perceptible to a subject; acquiring bioelectric signals from a subject via at least one sensor concurrently with the providing of the at least one stimulus; based on the bioelectric signals, determining a mental arousal score for the subject for the time duration; providing a virtual object in the virtual reality environment; and responsive to the mental arousal score, controllably varying at least one of the virtual object and the at least one stimulus. At least a part of the virtual object may be configured to be in a motion responsive to the mental arousal score, and wherein the controller is configured to controllably vary a speed or a frequency of the motion such that the speed or the frequency of the motion is a perceptible indicator of the mental arousal score. The controller may be further configured to provide audio feedback via the speaker to guide the subject towards a mental state that results in a reduction in the speed or the frequency of the motion. The controller may be configured to switch between providing an arousal-inducing stimulus and providing a relaxation-inducing stimulus, and wherein the audio feedback is provided concurrently with the relaxation-inducing stimulus.

### BRIEF DESCRIPTION OF DRAWINGS

FIGS. 1 and 2 are schematic diagrams of a mental arousal level regulation system according to an embodiment of the present disclosure.

FIGS. 3A and 3B schematically illustrate stimuli provided at a priming phase according to one embodiment of the present disclosure.

FIGS. 4A and 4B schematically illustrate stimuli provided according to a time-based control mechanism.

FIGS. 5A and 5B schematically illustrate stimuli provided according to a performance-based control mechanism.

FIG. 6 illustrates a virtual reality object according to an embodiment of the present disclosure.

FIGS. 7A and 7B show a part of a stimuli scheme used in experimental verification of the mental arousal regulation system of FIG. 2.

FIGS. 8 and 9 are plots showing the results obtained in the experiments.

### DETAILED DESCRIPTION

Reference throughout this specification to “one embodiment”, “another embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment. Furthermore, the described features, structures, or characteristics may be combined in any suitable manner in one or more embodiments. In the following description, numerous specific details are provided to give a thorough understanding of embodiments. One skilled in the relevant art will recognize, that the various embodiments be practiced without one or more of the specific details, or with other methods, components, materials, etc. In other instances, some or all known structures, materials, or operations may not be shown or described in detail to avoid obfuscation.

The word “exemplary” is used herein to mean “serving as an example, instance, or illustration.” Any embodiment described herein as “exemplary” is not necessarily to be construed as preferred or advantageous over other embodiments. As used herein, the singular ‘a’ and ‘an’ may be construed as including the plural “one or more” unless apparent from the context to be otherwise.

Terms such as “first” and “second” are used in the description and claims only for the sake of brevity and clarity, and do not necessarily imply a priority or order, unless required by the context. The terms “about” and “approximately” as applied to a stated numeric value encompasses the exact value and a reasonable variance as will be understood by one of ordinary skill in the art, and the terms “generally” and “substantially” are to be understood in a similar manner, unless otherwise specified.

As will be evident from the various non-limiting examples below, the present disclosure is applicable in a wide variety of situations, including but not limited to helping a subject learn how to regulate his/her own levels of mental arousal, helping a subject regulate mental arousal, or helping a subject achieve a target state of relaxation. The term “subject” may include but is not limited to: a patient with medical conditions relating to mental arousal, a person with non-medical condition relating to mental arousal but who may require external assistance for mental arousal regulation, or a person who is able to regulate his/her mental arousal without requiring external assistance.

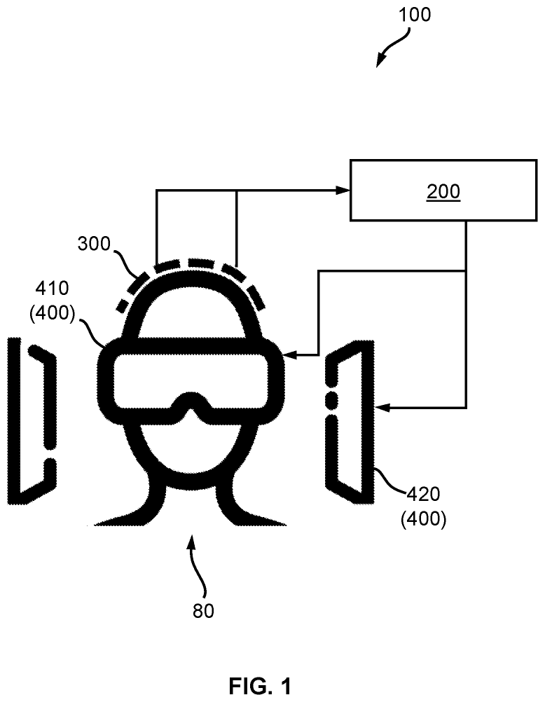

FIGS. 1 and 2 illustrate a system 100 configured to regulate mental arousal levels of a subject 80. The system 100 includes at least one sensor 300. For the sake of brevity, the singular term “sensor” as used herein may refer to one sensor or to a plurality of sensors being in use to collect data from one subject at any one time. The system 100 may be used with different types of sensors 300. For example, a heightened state of tension (e.g., mental arousal) in a subject 80 may manifest itself in the form of heart rate changes, body temperature changes, blood pressure changes, perspiration, facial expressions, etc. For the sake of brevity and for the purpose of illustration, the following will describe examples with reference to a multi-electrode electroencephalography sensing device or the like, such that bioelectric signals and/or electroencephalograms (EEG) can be acquired through EEG electrodes. In use, the sensor 300 may be worn or otherwise attached to the subject 80. For example, the sensor 300 may be configured as part of a wearable (such as a cap to be worn on the head of the subject).

The system 100 further includes a controller 200 in operable signal communication with the sensor 300 and with an interface 400. The interface 400 may include at least one visual device 410, at least one audio device 420, or a combination thereof. Examples of the at least one visual device 410 include but are not limited to virtual reality (VR) goggles, projectors, displays. Examples of the at least one audio device 420 includes but are not limited to speakers, earphones, etc. The interface 400 may be one hardware device with integrated audio-visual functions. The interface 400 may be configured to deliver a fully immersive VR experience to the subject 80, for example, through VR googles. The interface 400 may also be configured to deliver a VR experience that is less than fully immersive, in which the subject 80 remains cognizant of the actual physical environment. One example includes but is not limited to configuring a computing device to provide at least the controller 200 and a display (to serve as the interface 400).

It is understood that although the system 100 is described as including one controller 200, it is not outside the scope of the present disclosure for one or more embodiments described herein to be executed by more than one controller. The term “controller” is used herein in a general sense to refer to a processor or a programmable device configurable to execute code/programs/instructions. In some embodiments, the controller 200 may be embodied in a set of computer implementable instructions. The set of computer implementable instructions stored on a non-transitory computer readable medium of a computing device and executable by a processor in the computing device. In other embodiments, the controller 200 may be embodied in an integrated circuit which forms part of a device.

To aid understanding, various steps/inputs/outputs, etc., are represented in the form of a schematic block diagram in FIG. 2. It is understood however that any of the steps, etc., may be performed by one or more controllers by executing one or more pieces of code. The term “step” is used herein to refer generally to one or more steps, functions, routines, regimes, schemes, etc. As illustrated in FIG. 2, the controller 200 may include a pre-processing module 210, an arousal detection module 220, a stimuli switching control module 230, a feedback control module 240, and an interface controller/interface control module 250. The pre-processing module 210 may be configured to extract features based on bioelectric signals acquired via the sensor 300. The arousal detection module 220 may be based on a machine learning model, including but not limited to a convolution neural network. The feedback control module 240 is characterized in that the feedback control is personalized and in real-time. These and other features of the modules and method will be elaborated on below with the aid of non-limiting examples.

According to embodiments of the present disclosure, a method 700 of regulating levels of mental arousal may be described in terms of a priming phase 701 and a (real-time) guided task phase 702. In actual implementation, the priming phase 701 and the guided task phase 702 may be performed such that one phase 701/702 immediately follows after the other phase 702/701, or multiple sessions 750 of the guided task phase 702 may be performed after one session 750 of the priming phase 701. In some examples, the priming phase 701 and the guided task phase 702 may be performed at different times (e.g., on different days). Each priming phase 701 may include one or more cycles 710 of stimuli 720.

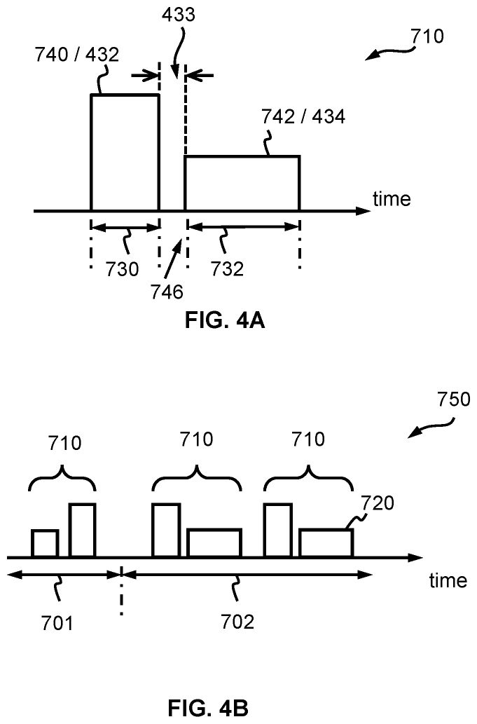

Each guided task phase 702 may include one or more cycles 710 of stimuli 720. Each “cycle” includes a first time duration 730 of a first stimulus 740 and a second time duration 732 of a second stimulus 742, in which the first stimulus 740 and the second stimulus 742 are associated with different levels of arousal of a mental state of a subject 80. In other words, each cycle 710 of stimuli is configured with a low arousal part (relaxation-inducing stimulus) and a high arousal part (arousal-inducing stimulus). In use, bioelectric signals 310 may be acquired from a subject 80 for a session 750 made up of a few cycles 710 of the priming phase 701 and a few cycles 710 of the guided task phase 702. The term “session” 750 as used herein refers to the subject 80 undergoing a priming phase 701, a guided task phase 702, or a combination of a priming phase 701 followed by a guided task phase 702. That is to say, the term “session” refers generally to a period when a subject 80 is being monitored and/or subject to stimuli by the system 100. The entire session 750 may take as little as 15 minutes, and the method of regulating mental arousal levels disclosed herein may be used by the subject 80 on a daily basis in the home environment without extensive disruption to the subject's daily routine. In some examples, multiple sessions 750 consisting of only the guided task phase 702 (e.g., FIGS. 5A and 5B) may be performed on different days over a period of weeks or months without the need for one or more intervening sessions of the priming phase 701. As will be clear from the following description, the system 100 enables personalized/customized configuration of the sessions for a subject 80 (e.g., number of cycles in the priming phase and/or the guided task phase, duration of each stimulus in a cycle, the conditions triggering a switch between the different stimulus, etc.) by the controller 200 based on bioelectric signals from the subject).

The following describes a method of priming the system 100. To illustrate the computational efficiency of the method of priming and not to be limiting, a session of priming may be completed in about 15 minutes or less in some examples. In the priming phase 701, the subject 80 is subjected to a selected series of stimuli 720 presented via the interface 400 such that the series of stimuli 720 is configured to be visible to or perceived by the subject 80 as part of a VR experience. At the same time as the presentation of the series of stimuli 720, bioelectric signals 310 are acquired from the subject 80 via the sensor 300. In other words, the bioelectric signals 310 are acquired concurrently or instantaneously with the presentation of each stimulus 720. The bioelectric signals 310 are processed by the pre-processing module 210 to remove noise and extract features. The arousal detection module 220 predictively determines whether the bioelectric signal 310 (acquired concurrently with a presentation of a stimulus 720) reflects a relaxed mental state of the subject or an aroused mental state of the subject 80 (at the time of perceiving the concurrently presented stimulus 720). The algorithm of the arousal detection module 220 is configured to generate a score to reflect a probability of an aroused mental state of the subject. This score (referred to as a mental arousal score 320) may be determined by the arousal detection module 220, in which the mental arousal score 320 correlates or is based on a probability of an aroused mental state of the subject 80.

The controller 200 may be configured with a control rule to determine if a switch of the stimulus is to be triggered. If the stimuli switching control module 230 determines that conditions for switching from one stimulus to another stimulus are satisfied, the stimuli switching control module 230 is configured to send a switching control signal to the interface control module 250. A two-way communication between the switching control module 230 and the feedback control module 240 enables both the control rule and the mental arousal score 320 to be taken as input to determine the stimulus to be presented next. In other words, the controller 200 may be in signal communication with the sensor 300 to receive bioelectric signals 310 of the subject 80, and configured to determine an arousal score 320 of the subject 80 based on the bioelectric signals 310. The controller 200 may be configured to control the interface controller 250 based on the arousal score 320 and a control rule, such that the interface 400 switches between presenting an arousal-inducing stimulus 432 and a relaxation-inducing stimulus 434.

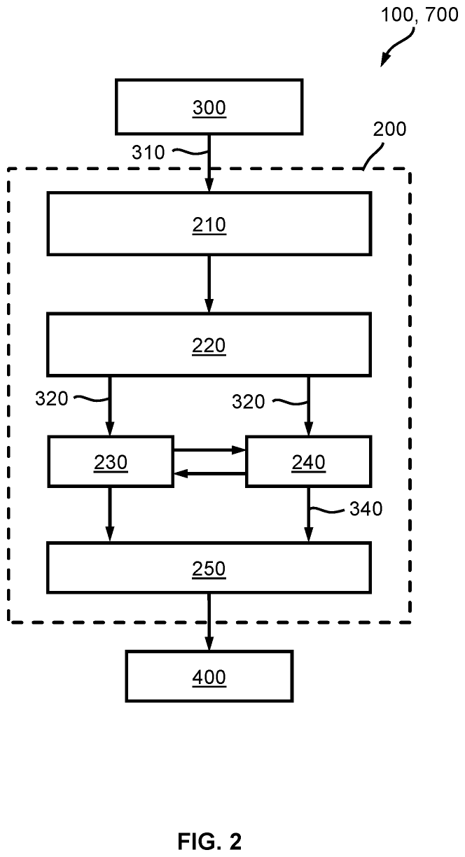

One cycle 710 of the stimuli presented in a priming phase 701 is schematically illustrated in FIG. 3A. Throughout the priming phase 701, the series of stimuli 720 may include at least one cycle 710 of at least two stimuli 720/742. Preferably, as schematically illustrated in FIG. 3B, the priming phase 701 includes multiple instances of switching 746 between two different stimuli, each stimulus being configured to induce a different level of mental arousal in the subject 80. In other words, in the priming phase 701, two or more calibration stimuli are provided to the subject 80, in which the calibration stimuli includes one or more arousal-inducing stimuli (high arousal stimuli 432) alternating with one or more relaxation-inducing stimuli (low arousal stimuli 434).

One method of triggering a switch 746 from one stimulus (a first stimulus 740) to another stimulus (a second stimulus 742) is referred to herein as a time-based control (TBC) mechanism. According to one example of the TBC mechanism applied in the priming phase 701, each cycle 710 includes one arousal-inducing stimulus (S2) 432 and one relaxation-inducing stimulus (S1) 434 in which both stimuli are presented to the subject for the same or substantially equal length of a time duration, tₜₐₛₖ. In one example, a first stimulus 740 is provided for a first time duration 730 of about one minute, followed by a second stimulus 742 which is provided for a second time duration 732 of about one minute. A break 433 (for example, a break duration of five seconds during which no stimulus is presented to the subject) may be provided between two sequential stimuli. The break duration is denoted by t_{break}. The TBC mechanism may be represented as follows:

$$Stimuli = \{ \begin{matrix}{S}_{1} & ( t < {t}_{task} ) \\ {S}_{break} & ( {t}_{task} < t < {t}_{task} + {t}_{break} ) \\ {S}_{2} & ( {t}_{task} + {t}_{break} < t < 2 {t}_{task} + {t}_{break} )\end{matrix} \qquad ( 1 )$$

where t is a time instant in each session.

The order in which the different stimuli are presented may differ from one priming session to another.

For the purpose of the present disclosure, the terms “arousal-inducing stimulus” 432 and “relaxation-inducing stimulus” 434 are used in a relative sense and with respect to a specific subject 80. For a particular subject, the arousal-inducing stimulus 432 refers to a stimulus which is found to likely induce a higher level of mental arousal in the subject 80, and the relaxation-inducing stimulus 434 refers to a stimulus found to likely induce a lower level of mental arousal in the subject 80. For different subjects at the same time or for the same subject at a different time, the absolute values of the arousal-inducing bioelectric signals and the relaxation-inducing bioelectric signals may be different. In this manner and in other aspects, the priming stage 701 and the method of mental arousal level regulation 700 is personalized to the individual subject 80.

At the end of a priming phase 701 for a subject, the arousal detection module 220 would be calibrated for the task phase 702. The arousal detection module 220 is calibrated to determine a mental arousal score 320 based on bioelectric signals 310 acquired during the task phase 702 from the same subject 80, in which the mental arousal score 320 is a personalized yet objective quantifiable measure of the subject's real-time state of mental arousal. The calibration priming 701 personalizes the classification model using data acquired from one subject such that the mental arousal scores 320 subsequently determined in the task phase 702 are indicative of how the same subject responds to stimuli. In other words, the mental arousal score 320 for a subject 80 is based on bioelectric signals 310 acquired exclusively from the same subject 80.

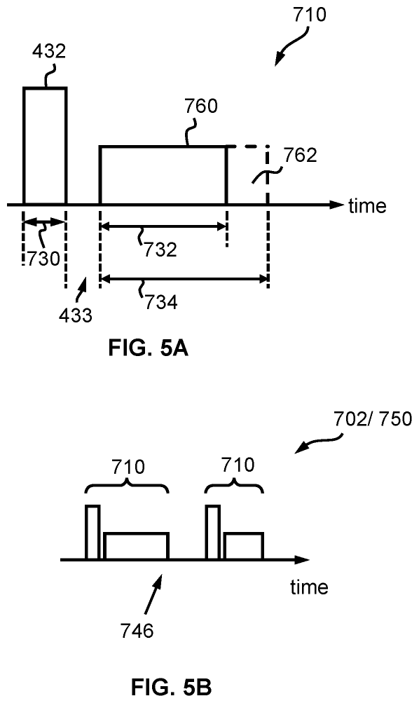

One example of the guided task phase 702 will now be described. The same system 100 (FIGS. 1 and 2) used for the priming phase 701 is used in the guided task phase 702. The system 100 is equipped with at least one sensor 300, an interface 400 configured to provide a virtual object 442 (FIG. 6) in a virtual reality environment 440 (the virtual object and the virtual reality environment are configured to be perceptible by the subject), and a controller 200 in operable signal communication with the at least one sensor 300 and the interface 400. The controller 200 is configured to perform a method 700 of mental arousal level regulation including: providing at least one stimulus 720 in the virtual reality environment 440 for a time duration; and acquiring bioelectric signals from the subject via the at least one sensor concurrently with the providing of the at least one stimulus. Based on the bioelectric signals 310, a mental arousal score 320 for the subject 80 for the time duration can be determined. The controller 200 is configured to be responsive to the mental arousal score 320, controllably varying at least one of the virtual object 442 and the at least one stimulus 720.

For the guided task phase 702, the sensor 300 is attached to the subject 80 in a manner similar to the priming phase 701, with the interface 400 configured to present selected stimuli visible to or perceptible by the subject 80. An EEG or bioelectric signals of the subject 80 are acquired from the at least one sensor 300. Preferably, the at least one sensor 300 is positioned to acquire bioelectric signals from the frontal area of the subject's brain.

The pre-processor 210 is configured to acquire the bioelectric signals 310 from the subject and to perform pre-processing of the bioelectric signals. Examples of pre-processing include but are not limited to the application of signal processing functions such as noise reduction and removal of artifacts. For example, a band-pass filter may be applied to remove noise from the bioelectric signals acquired. In one example, the band-pass filter is applied to remove frequencies below 0.3 Hertz and above 45 Hertz. Artifact removal may be carried out by the pre-processing module 210 by taking moving averages with multiple moving windows to obtain smoothed references, and subtracting the smoothed references from the EEG.

Let z ∈ [symbol] ^{L} denote the band-pass filtered bioelectric signal acquired from an EEG electrode (sensor) where L is the number of data samples. The windows to compute a set of moving averages are defined as lᵢ, i∈[1, m].

The moving averages are computed as:

$${z}_{( {l}_{i} )} [ t ] = \frac{1}{{l}_{i}} {\sum }_{t}^{t + {l}_{i}} z [ t ] \qquad ( 2 )$$

where t denotes samples of the data vector, for each window length lᵢ. The moving averages are subtracted from EEG and averaged as:

$$x = \frac{1}{m} {\sum }_{i = 1}^{m} z - {z}_{( {l}_{i} )} \qquad ( 3 )$$

To obtain artifact removed EEG. This step is repeated for the EEG from all electrodes, to obtain:

$$X = {[ {x}_{1} , … , {x}_{C} ]}^{T} ∈ {ℝ}^{C \times L} \qquad ( 4 )$$

where C is the number of electrodes (channels).

After artifact removal, the bioelectric signal is re-referenced to get the new representation, R=[r₁, r₂, . . . , r_{V}]^{T}, ∈ [symbol] ^{V×L} where V is the number of the re-referenced virtual channels. In order to train a classifier, the re-referenced bioelectric signal array is split into S short time segments, using a sliding window of length, L, and overlap, Lo, as Z⁽ⁱ⁾∈ [symbol] ^{S×V×L}^{_{w}}. For each segment, the EEG is then passed through N band-pass filters. Let the signal filtered in band b be f_{b}∈ [symbol] ^{L}^{_{w}}, and the sum of power in band b is calculated by:

$${F}_{b} = {\sum }_{i = 1}^{{L}_{w}} {f}_{{b}_{i}}^{2} \qquad ( 5 )$$

The relative power for all the bands is computed as:

$$\frac{{F}_{b}}{{\sum }_{b} {F}_{b}} \qquad ( 6 )$$

The relative power from all channels and bands constitutes the feature array F⁽ⁱ⁾∈ [symbol] ^{N×V}, i∈[1,S]. The feature segments array F∈ [symbol] ^{S×N×V} is then used for training a classification model (forming part of the arousal detection module 220). The feature segments used for personalizing the machine learning model all originate from the bioelectric signals acquired from one subject, and the machine learning model will be used for the same subject and no other subject. In other words, the machine learning model configured to predictively determine a mental arousal score 320 of the subject is essentially personalized according to bioelectric signals 310 acquired exclusively from the subject 80. This example of signal pre-processing by the pre-processing module 210 may also be used for pre-processing other physiological bio-signals 310.

The arousal detection module 220 may be configured with a machine learning model to classify whether the corresponding bioelectric signal reflects a relaxed mental state or an aroused mental state of the subject 80. The arousal detection module 220 is configured to output the classification result in the form of a mental arousal score 320. The mental arousal score 320 may reflect a probability of a mental arousal mental state of the subject. For example, the algorithm f may generate an arousal score Sₐᵣₒᵤₛₐₗ, which reflects the probability of the mental arousal state:

$${S}_{arousal} = f ( {seg}_{i} , Θ ) \qquad ( 7 )$$

where Θ is the model parameters learnt from training process, and segᵢ is the real-time segment of the pre-processed bioelectric signals. The mental arousal score 320 is input to both the feedback control module 240 and the stimuli switching control module 230.

The stimuli switching control module 230 is configured to determine when to end one stimulus (a first stimulus 740) and when to start another stimulus (a second stimulus 742). In other words, the stimuli switching control module 230 is configured to decide the interchange or switching of two modes of stimuli, namely, the relaxation-inducing stimulus (S1) 434 and the arousal-inducing stimulus (S2) 432. The stimuli switching control module 230 determines a switching between the two modes of stimuli based on output from the arousal detection module 220. The stimuli switching control module 230 is in signal communication with the interface 400 and controls the mode or type of stimuli provided to the subject: the arousal inducing stimuli 432 or the relaxation inducing stimuli 434. A two-way communication between the stimuli switching control module 230 and the feedback control module 240 determines the mode or type of stimuli provided to the subject. An output from stimuli switching control module 230 may be based on a control rule and the mental arousal score 320. The mental arousal score 320 of the subject may be continuously determined and monitored by the control rule and the type of stimuli 432/434 is switched or changed accordingly. Upon determining the mode of stimuli to present, a control signal (corresponding to the arousal regulation index 340) is sent to the interface controller 250 to control the interface 400. The guided task phase 702 may end according to a preset program in the control rule/controller.

In one embodiment of the present disclosure, the stimuli switching control module 230 is configured according to a time-based control (TBC) mechanism. According to another embodiment, the stimuli switching control module 230 is configured according to a performance-based control (PBC) mechanism.

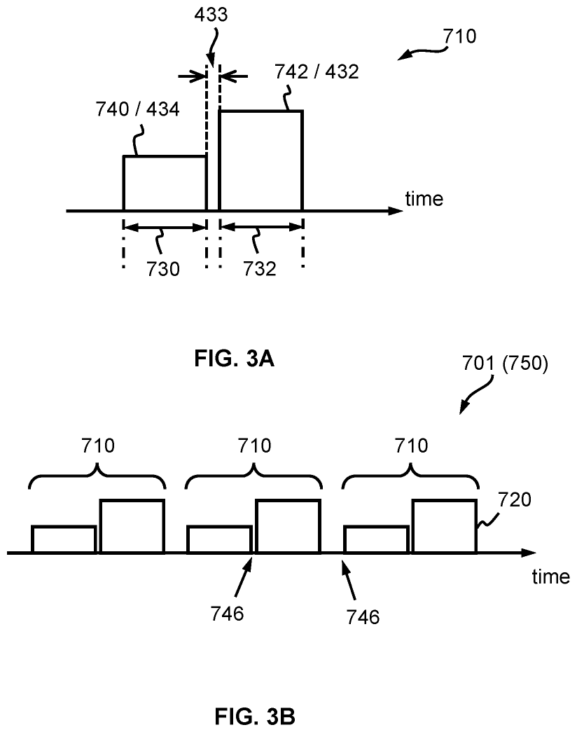

The TBC mechanism may be applied in either or both of the priming phase 701 and the guided task phase 702. An example of implementing the TBC mechanism at the guided task phase 702 is described with reference to FIGS. 4A and 4B. FIG. 4A schematically illustrates one cycle of stimuli 710, which is defined by one arousal-inducing stimulus 432 (in this example, the first stimulus 740) and one relaxation-inducing stimulus 434 (in this example, the second stimulus 742). The stimuli switching control module 230 is governed by a control rule that triggers a switch 746 from a first stimulus to a second stimulus when the first stimulus has been presented to the subject for a predetermined first time duration 730. As shown in FIG. 4B, the guided task phase 436 may include a plurality of cycles 710 of stimuli 720. According to the TBC mechanism, each cycle 710 is presented for a predetermined cycle duration, and then a switch is made to trigger a new cycle. Optionally, successive cycles or successive different modes of stimuli may be separated by a break 433.

For each cycle 710 of stimuli, the respective time durations 730/732 of the first stimulus 740 and the second stimulus 742 may be different, that is, tᵣₑₗₐₓ and tₐᵣₒᵤₛₐₗ are of different lengths of time, giving an asynchronous stimuli regime. The arousal-inducing stimulus 432 is selected or configured to evoke a more stressful or anxious mental state and to do so without causing harm to the subject. In one example, the control rule includes a maximum duration of the arousal inducing stimuli which is shorter than a maximum duration of the relaxation inducing stimuli.

The relaxation-inducing stimuli 434 may be configured to regulate the mental arousal level by natural neural feedback and mindfulness therapy. In another example, a duration of the arousal-inducing stimuli 432 is predetermined, and the control rule determines a duration of the relaxation-inducing stimuli 434 based on the arousal-inducing stimuli 432. In such a case, the duration of the arousal inducing stimuli 432 is proportional to a duration of the relaxation inducing stimuli 434, and may be defined as:

$${t}_{arousal} = θ {t}_{relax} \qquad ( 8 )$$

where θ is a proportionality coefficient. In some embodiments, the proportionality coefficient may be determined based on the bioelectric signals 310 acquired from the subject 80 in the priming phase 701.

As an alternative to the TBC mechanism, the PBC mechanism is characterized by a dynamic duration for each of the arousal/relaxation-inducing stimuli. The duration of each stimulus is determined by the stimuli switching control module 230 based on the subject's response to the (arousal-inducing or relaxation-inducing) and any resulting change in the subject's control of emotions or mental arousal. The subject's performance is quantified by an arousal regulation index (P) 340 which is calculated by the personalized feedback control module 240. The arousal regulation index 340 provides a measure or a value indicative of the level of mental arousal induced by the stimulus 432 during the arousal-inducing session, or indicative of the level of calm maintained or achieved by the subject when presented with a relaxation-inducing stimulus 434. The arousal regulation index 340 may be taken as a quantitative measure of the subject's ability to regulate his/her own level of mental arousal.

The stimuli switching control module 230 may be governed by a control rule that switches between different stimuli if the subject is able to maintain an arousal regulation index (P) that is higher than a certain threshold (T). As illustrated in FIG. 5A, if the arousal regulation index (P) acquired from the feedback control module 240 is found to be lower than the threshold (T), the stimuli switching control 230 continues the same/current stimulus 760 for an additional time, the current stimulus is continued for additional time (t_{expand}) 762 so that the subject 80 may have the additional time to make another attempt to regulate his/her own level of arousal. That is, in response to the controller finding the arousal regulation index 340 of the subject 80 to be below a threshold value, the controller 200 may be configured to increase a first time duration 732 of the current stimulus 760 by an additional duration 762 (total time duration 734), before switching over to the next stimulus. The controller 200 may be configured to determine an arousal regulation index 340 based on a number of instances when the mental arousal score is above an upper threshold or below a lower threshold. The threshold (T) is personalized or customized for each subject, and is based on the bioelectric signals acquired during the priming phase 701. For this reason, the priming phase 701 may also be referred to as the calibration phase.

$${t}_{session} = \{ \begin{matrix}{t}_{session} & ( P \geq T ) \\ {t}_{session} + {t}_{expand} & ( P < T )\end{matrix} \qquad ( 9 )$$

where t_{arousal session} is the initial duration of the arousal/relaxation-inducing session. FIG. 5B illustrate the guided task phase 702 in which the time duration required for the subsequent relaxation-inducing stimulus decreases, e.g., the subject 80 is better able to regulate his/her mental state from a highly aroused mental arousal state (high mental arousal score) to a less tense mental arousal state (lower mental arousal score) and thus require less time. Over time, the time duration patterns can help provide a consistent, quantifiable measure of improvement made by the subject in regulating his/her own mental arousal levels.

The feedback control module 240 of the system 100 is configured to perform any one or any combination of more than one of the following functions: (i) throughout a session, monitor the subject's state of arousal continuously while simultaneously evaluating the subject's arousal regulation index; (ii) enable the presentation of relaxation-inducing stimuli to the subject using a feedback that would appear natural (i.e., calming and non-provoking) and intuitive (i.e., user-friendly) to the subject who may be in a heightened state of arousal (e.g., mental arousal or stress); and (iii) based on the mental arousal scores, enable the subject to acquire an awareness of the subject's own state of mental arousal, and guide the subject into developing a mental habit of applying mindfulness therapeutic techniques.

The system 100 not only monitors the subject's mental state of arousal by acquiring bioelectric signals 310 continuously from the subject 80, but is also configured to determine an arousal regulation index (P) 340 based on the subject's mental arousal score Sₐᵣₒᵤₛₐₗ. The feedback control module 240 is configured to determine a personalized (i.e., subject-specific) arousal regulation index 340 that is applicable or customized to the particular subject.

In reality, different subjects find different situations stressful and different subjects may react with different degrees of arousal to the same arousal-inducing stimulus. One subject may find a particular technique helpful in calming down the mental state of arousal, while another subject may find another calming technique easier to apply. The personalized arousal regulation index determined on the basis of the personal mental arousal score of a subject enables a customized approach for each and every subject who uses the system 100.

The arousal regulation index 340 reflects the subject's mental state using a classification model personalized using the subject own's data/bioelectric signals (acquired during the priming phase 701 or from past arousal regulation sessions). Based on the mental arousal score Sₐᵣₒᵤₛₐₗ, a time phase evaluating (TPE) strategy may be determined to calculate the subject's arousal regulation index P. The arousal regulation index 340 indicates how much mental arousal is induced by a stimulus 720 during the arousal-inducing session 432 or how well the subject 80 is able to maintain calm mental state when presented with the relaxation-inducing stimulus 434

The time phase evaluating strategy of the mental arousal regulation index may be expressed as:

$${P}_{t} = {\sum }_{t}^{{T}_{session}} {t}_{task} - \frac{δ * t}{{T}_{session}} \qquad ( 10 )$$

where Pₜ is the arousal regulation index at time t, Tₛₑₛₛᵢₒₙ is the duration of the arousal regulation session, δ is the required duration when the subject needs to be in a mental state of arousal/relaxation, and tₜₐₛₖ is a subjective parameter, calculated as:
when the subject is presented with arousal-inducing stimuli 432:

$${t}_{task} = \{ \begin{matrix}1 & ( {S}_{arousal} > {ϕ}_{upper} ) \\ 0 & otherwise\end{matrix} \qquad ( 11 )$$

when the subject is presented with relaxation-inducing stimuli 434:

$${t}_{task} = \{ \begin{matrix}1 & ( {S}_{arousal} < {ϕ}_{lower} ) \\ 0 & otherwise\end{matrix} \qquad ( 13 )$$

where ϕᵤₚₚₑᵣ is the upper threshold of the mental arousal level, and ϕ_{lower} is the lower threshold of the mental arousal level. The upper threshold and the lower threshold are personalized parameters defined according to the subject's performance during the priming phase 701.

The feedback control module 240 is also configured to enable the presentation of relaxation-inducing stimuli 434 to the subject 80 using a feedback that would appear natural (i.e., calming and non-provoking) and intuitive (i.e., user-friendly) to the subject who may be in a heightened state of arousal. The feedback control module 240 of the system 100 is configured to present a virtual object 442 for the subject's viewing via the interface 400. The virtual object 442 is characterized by a variable property that corresponds to the subject's current level of mental arousal (e.g., mental arousal or stress). The feedback control module 240 is configured to change the variable property of the virtual object in accordance with the mental arousal score.

In one example, the feedback control module 240 is configured to present a VR image of a virtual object 442 (virtual reality object) in a VR environment 440, in which the variable property 446 of the object varies responsively to the mental arousal score acquired from the arousal detection module 220 (FIG. 6). In one example, the variable property is a flying height of the object in the VR environment 440. If the feedback control module 240 determines that the mental arousal score 320 of the subject 80 increases, the feedback control module 240 will case the virtual object 442 to appear to fly higher or to fly at a greater fly height (e.g., relative to a suitable virtual background/scene). If the feedback control module 240 determines that the mental arousal score 320 of the subject decreases, the feedback control module 240 will cause the virtual object to appear to fly lower or to fly at a lower fly height. By perceiving the flying height of the virtual object 442 over a given period of time, the subject 80 is able to concurrently monitor his/her own level of mental arousal in the given period of time, even in the absence of a trained therapist.

In another example, the virtual object 442 is a bird in flight, in which the variable property of the virtual object is a frequency of wing flapping 446 of the bird (FIG. 6). The feedback control module 240 may be configured to set a frequency of wing flapping 446 such that the frequency corresponds to the mental arousal score 320. The subject 80 is able to perceive an instantaneous (or near instantaneous) measure of his/her own level of mental arousal directly and easily, by simply looking at the virtual object's behavior, motion, state, and/or other variable property of the virtual object. In other words, at least a part 444 of the virtual object 442 may be configured to be in a motion responsive to the mental arousal score. The controller 200 may be configured to controllably vary a speed or a frequency of the motion such that the speed or the frequency of the motion is a perceptible indicator of the mental arousal score. The controller 200 may be configured to controllably vary a property 446 of the virtual object such that the property 446 tracks the mental arousal score 320. If the mental arousal score increases, the property also increases. If the mental arousal score decreases, the property also decreases. At least a part of the virtual object 444 may be configured to be in motion, the motion being characterized by a quantitative property perceptible by the subject, such that the quantitative property changes correspondingly with the mental arousal score 320 of the subject 80. The changes are substantially concurrent such that the subject 80 can obtain real-time update on his/her own state of mental arousal.

The system 100 provides the subject with a repeatable or objective measure of the subject's own level of (mental) arousal (e.g., mental arousal, stress, etc.). This repeatable or objective measure is personalized in that the priming, calibration or correspondence of a level of mental arousal of the subject to a variable property 446 of the virtual object 442 is based on the subject's own response or own state of arousal to selected stimuli 720. As described above, the mental arousal score of one subject 80 is based on bioelectric signals 310 from the same subject 80 and from no other subjects.

Alternatively described, the relaxation inducing stimuli 432 includes a first interaction. The first interaction may be a feedforward mechanism configured to inform the subject of his/her mental arousal score 320. For example, the first interaction includes a dynamic visual display 400, wherein the dynamic visual display 440 changes or moves according to the mental arousal score 320 of the subject. The virtual object 442 provides an instantaneous real-time mental arousal score 320 that would appear to be natural (i.e., calming, non-provoking, generally pleasant to see, etc.,) to the subject. Use of the system 100 would also appear intuitive to the subject 80 because there is no need for the subject to refer to a technical manual or to study neurotherapy in order to use the system or to understand the output (virtual object) correctly.

To perform this function, the feedback control module 240 is configured to translate the mental arousal score 320 (Sₐᵣₒᵤₛₐₗ) to a direct control signal, S_{feedback} to be provided to the interface controller, as:

$${S}_{feedback} = σ ( {S}_{arousal} ) \qquad ( 14 )$$

where σ(•) is a smoothing function, which provides a non-linear transformation to the mental arousal score to reduce or prevent sudden changes (such as increase/decrease) in the control signal. An example of the smoothing function is as follows:

$${S}_{feedback} = \frac{1}{1 + {e}^{- {S}_{arousal} + η}} \qquad ( 15 )$$

where e is the Euler's number, η is the bias coefficient. The control signal S_{feedback} controls the action of the virtual object.

The feedback control module 240 is further configured such that the system 100 can be used to guide the subject 80 into learning how to regulate his/her own levels of mental arousal. This function includes, based on the mental arousal scores, enabling the subject to acquire an awareness of the subject's own state of arousal, and guiding the subject 80 into developing a mental habit of applying calming techniques. This third aspect of the feedback control module 240 may be described as integrating reward-based neurofeedback strategy and mindfulness therapy with the mental arousal diagnostic function described above, in which mental arousal is one example of heightened mental arousal. The following describes a non-limiting example to illustrate this function.

In the example, the interface 400 includes a dynamic visual display 440 by which a virtual object 442 is presented to the subject 80. The virtual object 442 may be a virtual reality representation of a creature or an object of nature with elegant features (e.g., generally pleasant to look at). The virtual object 442 may be configured exhibit a motion 446 that can be modulated by the subject's mental arousal score or arousal mental state. For instance, the virtual object may be in the form of a bird 442 flying in a VR environment 440. A speed of the wings 444 flapping may be proportionate to the control signal S_{feedback}. For example, the speed of the wings flapping can be defined as:

$$Speed = ϵ * {S}_{feedback} \qquad ( 16 )$$

where E is the proportionate coefficient, and S_{feedback} is the control signal determined in equation (13). Therefore, the speed of the wings 444 flapping will be faster if the subject's mental arousal score is high.

An initial instruction of the system 100 may direct the subject to focus on the VR environment. One example of a script for the initial instruction may include: (i) “Find a comfortable, relaxed position. Tune in to breath or body sensations”; (ii)<pause for 10 seconds>; (iii) “Very slowly, when you feel ready, direct your attention to the environment and observe . . . ”.

The system 100 may provide a first duration of a high arousal stimulus to the subject 80. As the subject's level of mental arousal increases, the subject can instantaneously (simultaneously or in real-time) be aware of his/her own level of mental arousal increasing, simply by observing the VR object. The stimuli switching control module 230 triggers a switch of stimuli (according to any of the methods described above) such that subject is exposed to a second duration of a low arousal stimulus (relaxation-inducing stimulus).

Relative to the arousal-inducing stimulus 434, the relaxation-inducing stimulus 432 is one which has a lower probability of provoking a state of mental arousal or tension in the subject. For a subject 80 with few or no issues with mental arousal regulation, the subject's instantaneous or real-time mental arousal score during a low arousal part (relaxation-inducing stimuli) is expected to decrease or be relatively low (relative to the same subject's mental arousal scores in the presence of arousal-inducing stimuli). If the arousal detection module 210 determines that the subject's mental arousal score during the low arousal part of the guided task phase is relatively high or not decreasing, the system 100 provides a second interaction to guide the subject 80 into thoughts or behavior that may help to lower the mental arousal score 320 (regulate mental arousal level).

The second interaction may be an audio feedback 420 or a voice guided feedback which is determined based on the mental arousal regulation index P of the subject 80. The audio feedback 420 may be played in specific intervals and/or has a gentle and calming voice. The audio feedback 420 may also be a gentle audio guidance as a form of mindfulness therapy. For instance, if the subject 80 is learning to relax, the voice guided feedback informs the subject their progress and compliments the subject. In other words, the controller 200 may be further configured to provide audio feedback 420 via a speaker to guide the subject 80 towards a mental state that results in a reduction in the speed or the frequency of the motion. The controller 200 may be configured to switch between providing an arousal-inducing stimulus 432 and providing a relaxation-inducing stimulus 434, with the audio feedback 420 being provided concurrently with the relaxation-inducing stimulus 434.

If the subject's arousal score is high or increasing, the audio feedback 420 may guide the subject 80 to focus on the act of breathing and to control his/her breathing before redirecting his/her attention to the VR environment 440. An example of a script for the audio feedback 420 for a subject 80 with mental arousal issues may include: (i) “See if you can feel the sensations of your breath, taking one breath at a time. When one breath ends, the next breath begins.”; (ii)<pause for 10 seconds>; (iii) “Now slowly tune in to your environment.”. In another example, the audio feedback may attempt to reassure the subject and to redirect the subject's attention to his/her breathing, such as: “You may find your mind starting to wonder, or your mental arousal (level of tension) increasing. If that happens, it is not a problem. Gently redirect your attention to your breath, and slowly direct your attention back to the environment.”. In yet another example, the audio feedback may attempt to reassure the subject, such as: “Notice yourself getting lost in thought and returning to your breath and environment again and again. See if you can be really kind to yourself in this process.”.

If the subject is able to regulate mental arousal well, the system 100 may be configured to “reward” the subject. On the other hand, if the system 100 detects that the subject is possibly bored or sleepy (e.g., the subject may be overly calm), the audio feedback 420 may attempt to bring their attention back to the VR environment 440. Examples of such audio feedback 420 may include: “Feel the sensations of your breath, one breath at a time. Slowly, tune in to your environment.”. Another example is: “If you feel sleepy or when your mind starts to wonder, it is not a problem. Just notice what happens, and very gently redirect your attention back to the environment.”. The audio feedback 420 may also attempt to increase the mental arousal score of the subject, such as: “Observe yourself getting lost in thought and returning to your environment again and again. See if you can bring a soft, curious attention to your environment.”. The audio feedback 420 is responsive to the real-time instantaneous mental state of the subject 80, and is sensitive enough to differentiate at least broadly between a state of being overly calm, a state of mental arousal appropriate to the stimulus being presented, and an overly aroused mental state (e.g., mental arousal, tension, stress, etc.) for the stimulus being presented at the time. The arousal scoring method is personalized at least at the priming phase 701 so that the mental arousal regulation index P is a personalized index that enables the system 100 to distinguish between an appropriate level of arousal and an inappropriate level of arousal (relative to the stimuli to which the subject is reacting to).

At this junction, it may be useful to clarify that it is healthy for a subject to respond with a heightened state of tension when the subject is exposed to a high arousal stimulus. It is therefore not the intent of the audio feedback 420 of the system 100 to calm the subject down whenever the arousal score of the subject is considered “high”. In this aspect at least, the audio feedback 420 of the present disclosure is clearly distinguishable from simply playing “calming music” or a voice track repeating instructions for deep breathing/relaxation exercises regardless of the real-time mental state of the subject. In fact, as described, where required, the subject 80 is “awakened” by the system 100. In other words, the audio feedback 420 may be configured or selected responsive to the mental arousal score 320, such that it helps to direct the subject 80 towards changing his/her own mental arousal score 320 towards a preferred range of values.

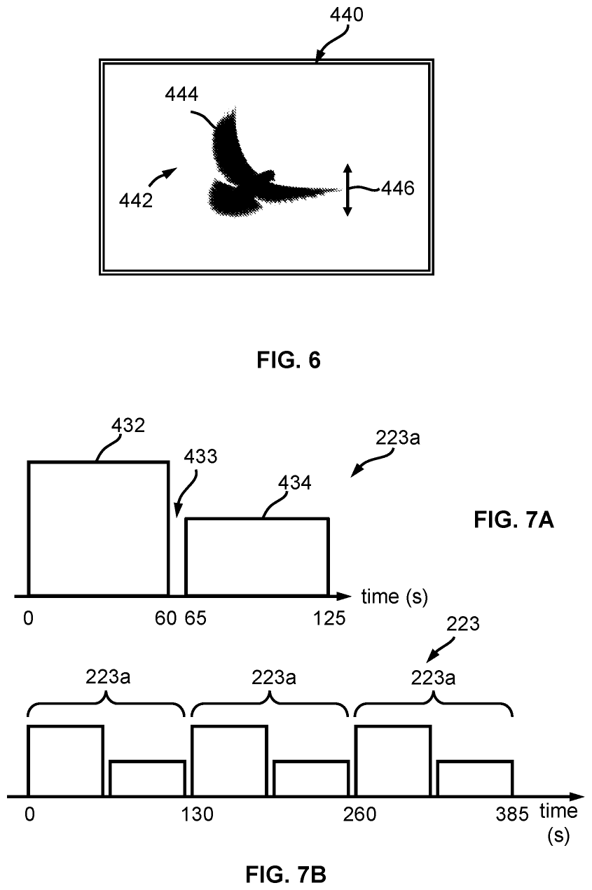

One embodiment of the system and mental arousal regulation method was experimentally verified. Eighteen subjects were recruited, and each subject underwent a priming phase (calibration regime 223). FIGS. 7A and 7B show a part of the stimulus scheme used in the experiments. The priming phase 223 includes three cycles of stimuli 223a. Each cycle of stimuli 223a includes one arousal-inducing stimuli (high arousal part) 432 and one relaxation-inducing stimuli (low arousal part) 434, each stimuli (part) 432/434 being sustained for a duration of 60 seconds. Each cycle 223a lasted for 125 seconds with a 5-seconds break 433 in between the mental arousal-inducing part and the relaxation-inducing stimuli part 432/434.

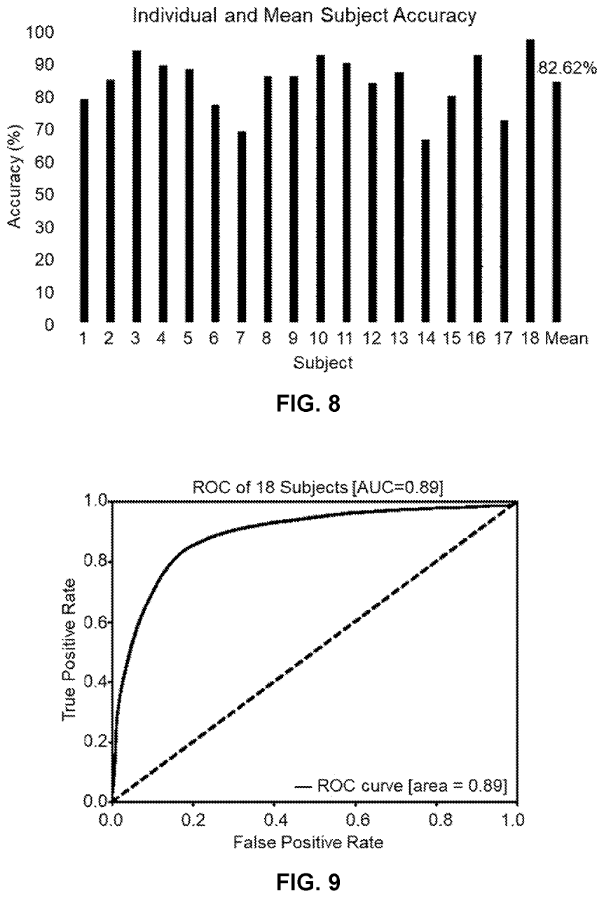

After acquiring the bioelectric signals (also referred to as the calibration signals) during the priming phase, the arousal detection method was applied to train a classifier (classification machine learning model) specific to each subject. The classifier is configured to classify an arousal score of the subject into one of two arousal states: high and low. A “leave one cycle out” cross-validation is applied to evaluate the performance of the classifier. The results are shown in FIG. 8 and FIG. 9. The average accuracy for all the subjects is at 82.62%, with the average area under curve (AUC) of the receiver operating characteristic (ROC) curve being 0.89 for all subjects combined. This indicates that the classifier algorithm of the arousal detection module 210 performs well in distinguishing between the two arousal states. Table 1 shows a comparison among different machine learning methods. It is shown that the proposed mental arousal regulation method achieves the highest classification accuracy for arousal/emotion detection. The results are encouraging and indicates that the mental arousal regulation system 100 is capable of distinguishing between different levels of mental arousal or tension in the mental state of subjects.

TABLE 1
Accuracy comparison among different methods.

| | | Standard |
|---|---|---|
| Method | Accuracy (%) | Deviation (%) |
| The proposed arousal detection method | 82.62 | 9.58 |
| Support Vector Machine (DE) | 80.72 | 10.11 |
| Support Vector Machine (RP) | 78.80 | 8.77 |
| EEGNet | 80.18 | 12.44 |
| Deep Convolutional Neural Network | 80.17 | 10.61 |
| (DeepConvNet) | | |

The present disclosure includes a machine learning based system 100 and method 700 for mental arousal regulation which provides a subject 80 with both arousal-inducing stimuli 432 and relaxation-inducing stimuli 434. The arousal-inducing stimuli 432 is selected to be one that is likely to raise a mental arousal level in the subject 80 without exceeding a threshold (beyond which the subject may be harmed). The arousal-inducing stimuli 432 is alternated with relaxation inducing stimuli 434. The bioelectric signals 310 continually being acquired from the subject 80, and the stimuli 720 provided at the time of acquiring the bioelectric signals, form the basis of an arousal detection method 700. The arousal detection method 700 provides an instantaneous or near instantaneous prediction of the level of mental arousal of the subjects 80. By being exposed to alternating modes of stimulus which would have elicited different levels of mental arousal response, a subject can be safely guided to experientially try out different ways to regulate his/her own level of mental arousal until the subject 80 can regulate appropriate levels of mental arousal appropriate to the different modes of stimuli. The instantaneous or real-time feedback 442 (visually perceptible by the subject himself/herself while undergoing mental arousal regulation) provides a personalized yet objective quantifiable measure of the subject in relation to known modes of stimuli. All these provide an advantage over conventional neurofeedback therapy that do not teach the subject to self-regulate mental arousal levels.

In addition, the objective quantifiable measure of how the subject 80 handles different modes of stimuli enable the performance of the subject to be tracked in a meaningful manner over a relatively long period of time (e.g., weeks, months, years, etc.). At the same time, each session of mental arousal regulation may take as little as 10 to 15 minutes, and can be carried out with minimal disruption to the subject's daily routine.

The objective quantifiable measure of the subject's current state of mental arousal also helps to address problems with subjects not being able to accurately recall or articulate how they felt in a particular situation, especially when asked to describe their reactions or feelings to a past event. Some subjects may feel too uncomfortable describing their anxieties to another person, even if the other person is a professional therapist. It is also doubtful if subjects can accurately gauge their own feelings when they are in a highly anxious mental state or even in an overly calm (e.g., depressed) mental state.

As can be understood from the examples given above, the mental arousal regulation system 100 is configured to provide a guided mental arousal regulation process 700. The mental arousal regulation system 100 is guided in the sense that it may be configured to operate as intended either within or outside a clinic or healthcare facility. The mental arousal regulation system 100 may be used by the subject (the patient) in an environment in which the subject feels safe and/or comfortable, even if it involves the therapist being in a different physical location as the subject. It can be further understood from the above examples that the mental arousal regulation system 100 is configured to equip the subject with moment-to-moment awareness of his/her own state of mind, such that the subject can practice mindfulness therapy with the aid of the same mental arousal regulation system 100. The mental arousal regulation system is configurable to be fully self-guided so that the subject can learn to regulate mental arousal in a safe and comfortable environment without fear of being judged by other people. In some examples, the method may be implemented with the aid of a computing device with a processor or controller and a non-transitory computer readable medium having stored thereon computer implementable instructions executable by the controller. The computing device may be coupled with a speaker for delivering the audio feedback and a (virtual reality or graphical) interface for presenting the virtual object and/or the stimuli in a fully-immersive VR environment or a partial VR environment.

All examples described herein, whether of apparatus, methods, materials, or products, are presented for the purpose of illustration and to aid understanding, and are not intended to be limiting or exhaustive. Various changes and modifications may be made by one of ordinary skill in the art without departing from the scope of the invention as claimed.

1. A system for mental arousal level regulation of a subject, the system comprising:
at least one sensor;
an interface configured to provide a virtual object in a virtual reality environment, the virtual object and the virtual reality environment being perceptible by the subject;
a controller in operable signal communication with the at least one sensor and the interface, the controller being configured to perform a method of mental arousal level regulation including:
providing at least one stimulus in the virtual reality environment for a time duration;
acquiring bioelectric signals from the subject via the at least one sensor concurrently with the providing of the at least one stimulus;
based on the bioelectric signals, determining a mental arousal score for the subject for the time duration; and
responsive to the mental arousal score, controllably varying at least one of the virtual object and the at least one stimulus.

2. The system according to claim 1, wherein the controller comprises a machine learning model configured to predictively determine the mental arousal score, and wherein the machine learning model is personalized according to bioelectric signals acquired exclusively from the subject.

3. The system according to claim 2, wherein the machine learning model comprises a classification model trained using feature segments originating from the bioelectric signals acquired exclusively from the subject.

4. The system according to claim 1, wherein the controller is configured to controllably vary a property of the virtual object such that the property tracks the mental arousal score.

5. The system according to claim 1, wherein at least a part of the virtual object is configured to be in motion, the motion being characterized by a quantitative property perceptible by the subject, such that the quantitative property changes correspondingly with the mental arousal score of the subject.

6. The system according to claim 1, wherein the controller is configured to controllably vary the at least one stimulus provided in the virtual reality environment by switching between providing a first stimulus and providing a second stimulus.

7. The system according to claim 6, wherein the controller is configured to determine respective mental arousal scores for the first stimulus and the second stimulus based on the bioelectric signals from the subject, and wherein the respective mental arousal scores are indicative of different mental arousal levels with respect to the subject.

8. The system according to claim 6, wherein with respect to the subject, the first stimulus is an arousal-inducing stimulus and wherein the second stimulus is a relaxation-inducing stimulus.

9. The system according to claim 6, wherein with respect to the subject, the first stimulus is a relaxation-inducing stimulus and wherein the second stimulus is an arousal-inducing stimulus.

10. The system according to claim 6, wherein the controller is configured to switch from providing the first stimulus to providing the second stimulus after providing the first stimulus for a first duration.

11. The system according to claim 10, wherein the first duration is at least a predetermined duration of time.

12. The system according to claim 1, wherein the controller is configured to determine the mental arousal score based on the bioelectric signals acquired exclusively from the subject.

13. The system according to claim 1, wherein the controller is configured to determine an arousal regulation index based on the bioelectric signals, the arousal regulation index being indicative of a level of mental arousal induced in the subject.

14. The system according to claim 13, wherein the arousal regulation index is configured to be a quantitative measure of the subject's ability to regulate the subject's own level of mental arousal.

15. The system according to claim 13, wherein in response to the controller finding the arousal regulation index of the subject to be below a threshold value, the controller is configured to increase the first duration by an additional duration.

16. The system according to claim 13, wherein the controller is configured to determine a mental arousal regulation index based on a number of instances when the mental arousal score is above an upper threshold or below a lower threshold.

17. The system according to claim 1, wherein the controller is further configured to provide an audio feedback responsive to the mental arousal score, and wherein the audio feedback is configured to direct the subject towards changing the mental arousal score towards a preferred range of values.

18. A method of regulating a mental arousal level of a subject, the method comprising using the system as recited in claim 1 to:
provide at least one stimulus in a virtual reality environment for a time duration;
acquire bioelectric signals from the subject via at least one sensor concurrently with the providing of the at least one stimulus;
based on the bioelectric signals, determine a mental arousal score for the subject for the time duration; and
controllably vary at least one of a virtual object and the at least one stimulus, the controllably varying being responsive to the mental arousal score, at least one sensor, wherein the virtual object is provided in the virtual reality environment, the virtual object and the virtual reality environment being configured to be perceptible by the subject.

19. A computing device comprising: a controller; a speaker coupled to the controller; an interface coupled to the controller; and a non-transitory computer readable medium having stored thereon computer implementable instructions executable by the controller, the controller being configured to perform a method of mental arousal level regulation including:
providing at least one stimulus as a virtual reality environment via the interface for a time duration, the virtual reality environment being perceptible to a subject;
acquiring bioelectric signals from a subject via at least one sensor concurrently with the providing of the at least one stimulus;
based on the bioelectric signals, determining a mental arousal score for the subject for the time duration;
providing a virtual object in the virtual reality environment; and
responsive to the mental arousal score, controllably varying at least one of the virtual object and the at least one stimulus.

20. The computing device according to claim 19, wherein at least a part of the virtual object is configured to be in a motion responsive to the mental arousal score, and wherein the controller is configured to controllably vary a speed or a frequency of the motion such that the speed or the frequency of the motion is a perceptible indicator of the mental arousal score,
wherein the controller is further configured to provide audio feedback via the speaker to guide the subject towards a mental state that results in a reduction in the speed or the frequency of the motion, and
wherein the controller is configured to switch between providing an arousal-inducing stimulus and providing a relaxation-inducing stimulus, and wherein the audio feedback is provided concurrently with the relaxation-inducing stimulus.

21-22. (canceled)
